(19) United States
(12) Patent Application Publication
(10) Pub. No.: US 2010/0258134 A1
(43) Pub. Date: Oct. 14, 2010

(54) ENDOTRACHEAL TUBE HAVING IMPROVED SUCTION LUMEN

(75) Inventors: Joel C. Colburn, Walnut Creek, CA (US); Douglas A. Clement, Livermore, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/821,018

(22) Filed: Jun. 22, 2010

Related U.S. Application Data
(63) Continuation of application No. 11/579,995, filed on Nov. 10, 2006.
(60) Provisional application No. 60/570,171, filed on May 12, 2004.

(51) Int. Cl. A61M 16/04; B32B 37/12

(52) U.S. Cl. 128/207.15; 156/293

(57) ABSTRACT

A surgical/medical tube such as an endotracheal tube 10 is provided having a tubular body 12 equipped with an inflatable cuff 22 and respective, separate cuff inflation and fluid removal lumens 24, 30. A fluid removal opening 32 communicates with lumen 30 and is located proximal to cuff 22. A projection 38, 38a, 40, 42 is located in close proximity to the opening 32 and is configured to prevent contact between the opening 32 and the adjacent tracheal wall.

### CROSS REFERENCED TO RELATED APPLICATION

This application claims the benefit of Provisional Application of Ser. No. 60/570,171 filed on May 12, 2004. This Provisional Application is incorporated by reference herein.

### BACKGROUND OF THE INVENTION

1. Field of the Invention

The present invention is broadly concerned with improved surgical/medical tubes, and particularly inflatable cuff-type endotracheal tubes adapted to be intubated into the trachea of a patient to facilitate mechanical ventilation of the patient's lungs. Certain embodiments include tubes having improved suction apparatus for periodic removal of fluids which collect adjacent the cuff. The tubes may include a strategically located projection proximal to the suction fluid opening that prevents contact between the suction opening and the tracheal wall to reduce or eliminate possible occlusion of the opening and prevention of fluid removal.

2. Description of the Prior Art

Conventional methods of endotracheal intubation involve the insertion of a tubular device such as an endotracheal tube into the trachea. The endotracheal tube passes through the trachea and terminates above the carina allowing gases to be directed through the tubes and into the lungs.

A primary objective of this treatment is the mechanical ventilation of a patient's lungs, which may be required owing to the patient's disease or injury. In order to create the air pressure necessary to artificially ventilate the lungs, the passageway around the tube must be sealed. This is accomplished through use of an inflatable cuff provided around the tube. With the tube in place, the cuff is typically located about 3-5 centimeters above the carina and within the trachea. The cuff is inflated to expand and seal against the wall of the trachea, thereby preventing gases that are being pumped into the lungs from backing up around the tube.

While this method of treatment has been quite successful, problems remain. For example, cuffed endotracheal tubes can present a problem in that secretions produced above the cuff in the trachea are prevented from flowing along the channel and will thereby collect above the cuff, providing a site for the possible accumulation of pathogens.

Various methods have been devised for removing such secretions. For example, one or more small apertures may be provided above the cuff with an associated suction lumen. Accordingly, fluids can be periodically or continuously removed through the opening and lumen by suction. Alternatively, a cuffed lumen may have a suction opening oriented so that a portion of the cuff folds back towards the opening in order to facilitate fluid removal.

It is generally believed that cuffed endo tracheal tubes are effectively centered within the trachea upon inflation of the cuff, so that the suction opening is spaced from the tracheal wall. However, contrary to this belief, it is now been found that endotracheal tubes do not necessarily self-center upon cuff inflation. Occasionally, owing to the curvature thereof, the suction opening may locate very near the tracheal wall. If this occurs, it may be possible that a suction opening actually contacts the tracheal wall, whereby application of a vacuum can cause the tracheal wall membrane to be drawn into the suction opening, thereby occluding it. This condition may prevent the proper removal of secretions from the subglottic space and may also cause trauma to the tracheal wall.

There is accordingly a need in the art for an improved surgical/medical tube such as an endotracheal tube which is specifically designed to prevent contact between the fluid removal opening and portions of the adjacent body cavity wall.

### SUMMARY OF THE INVENTION

In accordance with one aspect of the present invention, there is provided improved surgical/medical tubes, and especially endotracheal tubes, which are designed to prevent inadvertent contact between the fluid removal openings thereof and adjacent tissues.

Broadly speaking, surgical/medical tubes in accordance with the present exemplary embodiments, generally include an elongated, gas-conveying tubular body, and an inflatable sealing cuff mounted on the body and adapted to seal against the wall of a body cavity. Such tubes may be equipped with a suction lumen extending along the tube and terminating in a suction opening adjacent the cuff for permitting suction removal of collected fluids in the region of the cuff. A projection may be mounted on the exterior of the body in closely spaced relationship to the opening, with the projection configured to prevent contact between the suction opening and the body cavity wall.

The tube assembly may be specifically designed as an inflatable cuff-type endotracheal tube, with a projection located in close proximity to the suction opening in order to prevent contact between the opening and the tracheal wall. Such a projection may be in the form of an elongate or round body, a transversely mounted O-ring or tubing section, or collar or mini-cuff, for example.

### BRIEF DESCRIPTION OF THE DRAWINGS

FIG. 1 is an elevational view of an endotracheal tube in accordance with an exemplary embodiment of the invention, shown with the cuff thereof in an inflated condition;

FIG. 2 is an enlarged, fragmentary view of the endotracheal tube, specifically at the region of the fluid removal opening adjacent the cuff;

FIG. 3 is a sectional view taken along line 3-3 of FIG. 2;

FIG. 4 is an enlarged, fragmentary view similar to that of FIG. 2, but illustrating another embodiment of the invention;

FIG. 5 is a sectional view taken along line 5-5 of FIG. 4;

FIG. 6 is an enlarged, fragmentary view similar to that of FIG. 2, but illustrating a still further embodiment of the invention;

FIG. 7 is an enlarged fragmentary view depicting another embodiment of the invention; and

FIG. 8 is a view in partial vertical section of the embodiment of FIG. 7, illustrating the internal construction thereof.

FIG. 9 is an enlarged, fragmentary view similar to that of FIG. 4, but illustrating another embodiment of the invention.

### DETAILED DESCRIPTION OF THE PREFERRED EMBODIMENT

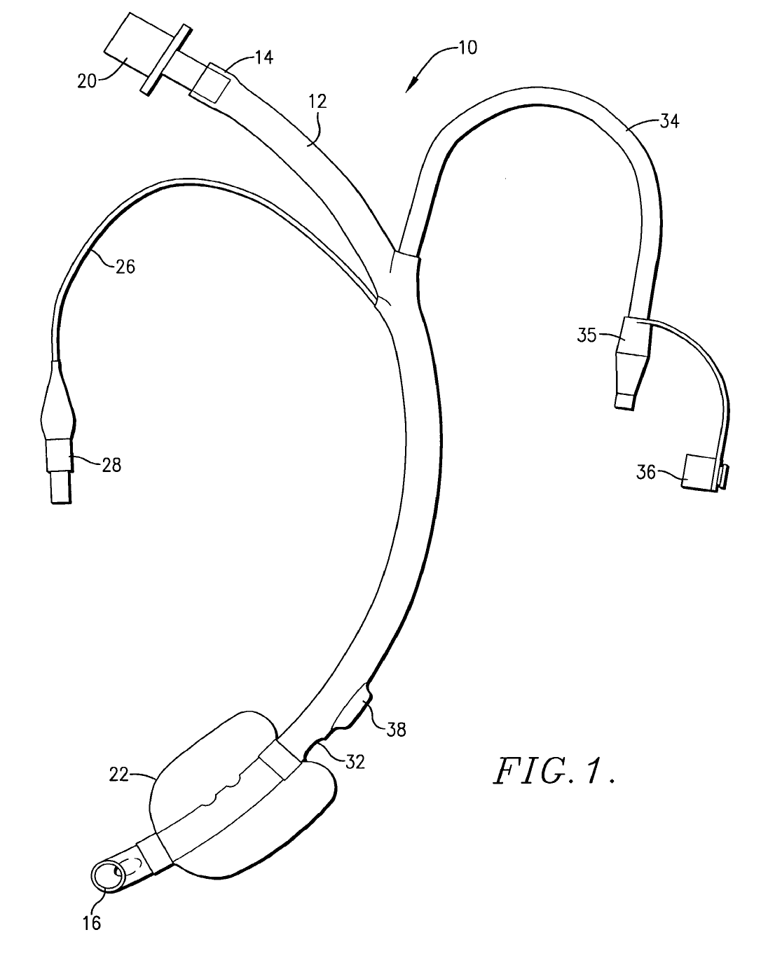

Turning now to the drawing, an exemplary endotracheal tube 10 is depicted in FIG. 1. The tube 10 includes a primary tubular body 12 having opposed, open proximal and distal ends 14 and 16. The body 12 defines a central gas-conveying passageway 18 for mechanical ventilation of a patient. The proximal end 14 is equipped with a connector 20 and, in use, the connector 20 is designed for attachment to a mechanical ventilator (not shown).

The overall tubular body 12 further includes an inflatable resilient cuff 22 adjacent the distal end thereof. During intubation of the tube 10, the cuff 22 is collapsed. However, once properly in place, the cuff 22 is fully inflated via lumen 24 formed in body 12 and having a connected proximal inflation line 26 terminating in a fixture 28 allowing such cuff inflation.

The tubular body 12 also includes a fluid removal lumen 30 situated in opposed relation to lumen 24 and likewise formed in the wall of the body 12. The lumen 30 terminates in an opening 32 extending wholly through the wall of body 12 and positioned above (e.g., at least about ⅛ inch) the proximal end of cuff 22. As shown, an exterior suction tube 34 is also provided which is in communication with lumen 30. The tube 34 has an endmost fixture 35 including a cap 36.

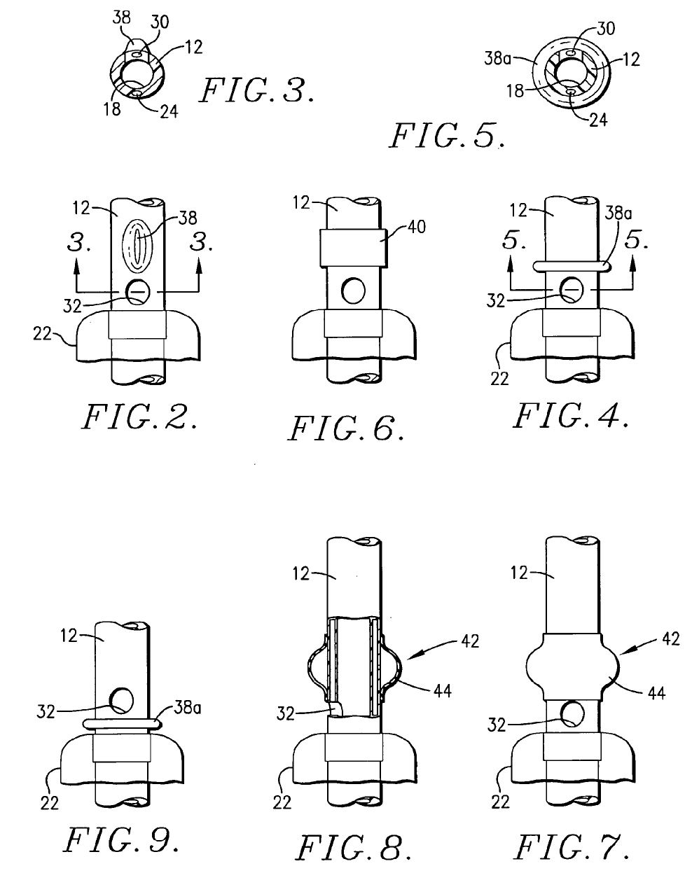

The tubular body 12 is also equipped with a projection 38 in close proximity to opening 32 in order to prevent contact between the latter and the patient's tracheal wall. In the embodiment of FIGS. 1-3, the projection 38 is oblong or oval-shaped, with the longitudinal axis thereof generally parallel with the axis of body 12. The projection 38 may extend outwardly from the adjacent exterior surface of the body 12 a distance of from about 0.030-0.080 inches, such as in a range from about 0.040-0.060 inches, for example.

In an alternate embodiment (see FIGS. 4-5), a projection 38a is provided, here in the form of an O-ring extending transverse to the axis of body 12. The effective height of the ring 38a relative to the exterior surface of tubular body 12 may be the same as that for the projection 38. Moreover, while not shown, it will be appreciated that the ring 38a need not extend wholly around the body 12, but it generally does for ease of manufacture. In another embodiment, a short stretch of shrink tubing 40 is applied to the body 12 and heated to secure the tubing to the body (see FIG. 6).

Referring to FIGS. 7-8, a projection 42 may be provided in the form of a small, secondary inflatable cuff 44 which is affixed to tubular body 12 slightly above opening 32. The cuff 42 is secured to the body 12 by adhesive or by any conventional means, and may be inflated via lumen 24 or by provision of any entirely separate lumen. Also, the cuff 44 may extend entirely about body 12, or the cuff may only extend in the area above opening 32, without extending fully about body 12, for example.

Referring to FIG. 9, an alternate embodiment is shown wherein projection 38a is again provided, here in the form of an O-ring extending transverse to the axis of body 12, but disposed below or distally from opening 32. As shown in FIG. 9, projection 38a can be between inflatable cuff 22 and opening 32. While the alternative embodiment of FIG. 9 is shown with ring 38a below opening 32, any of the disclosed projections 38 could be so disposed distally from opening 32.

To place the tube 10, first the epiglottis is lifted and the tube 10 is inserted down the trachea to a point just above the carina. The cuff 22 is then inflated by pumping air into the cuff, this being accomplished through the tube 26 and lumen 24. Typically, inflation air is provided by a syringe inserted into fixture 28. In any case, inflation of the cuff 22 to 25-30 cm H₂O (or other clinically appropriate pressure level) effects sealing of the trachea. The proximal end 14 of tubular body 12 can then be attached to a ventilator for mechanical ventilation of the patient by means of connector 20. Following intubation, fluid secretions may begin to build up at the proximal end of cuff 22. These secretions may carry bacteria or other pathogens in an environment ideal for pathogen growth. Accordingly, the secretions may be periodically or continuously removed through suction opening 32 and lumen 30. To this end, the cap 36 is removed and fixture 35 may be connected to a suction machine (not shown) for fluid removal; alternately, a syringe may be used for this purpose. Accordingly, the incidence of fluid leakage is reduced.

It will moreover be appreciated that the provision of the projection 38, 38a, 40, or 42 (or any other suitable projection) effectively prevents contact between the suction opening 32 and the adjacent tracheal wall. This is true even if the tube 10 is slightly out of place or positioned off-center relative to the trachea. Thus, if the tubular body 12 is located in a position which would otherwise permit contact between the suction opening 32 and the tracheal wall, the projection comes into play to prevent such contact. Consequently, even under such circumstances, the tube 10 continues to operate normally for the important removal of collected secretions above cuff 22.

It will be appreciated that while the invention is particularly described in the context of an endotracheal tube, the invention is not so limited. Thus, the same principles may be applied to a variety of other surgical/medical tubes equipped with inflatable cuffs. Additionally, while two specific embodiments of contact-preventing projections have been illustrated and described, a variety of other projection shapes and sizes can be used to good effect.

What is claimed is:

1. A medical tube comprising:
a body comprising an elongated gas-conveying tube having a distal end configured to be placed in a body cavity of a patient and a proximal end configured to extend outwardly from the patient, the distal end having a distal opening and the proximal end having a proximal opening;
a sealing cuff mounted on the body proximal to the distal opening, wherein the sealing cuff is configured to seal against the wall of the body cavity;
a suction lumen extending along the body and terminating in a suction opening adjacent and proximal to the sealing cuff, and wherein the suction lumen and suction opening are configured to permit suction to remove substances from the body cavity; and
a projection configured to prevent occlusion of the suction opening, wherein the projection is located on the exterior of the body adjacent and proximal to the suction opening, and wherein the projection is configured not to seal against the wall of the body cavity.

2. The tube of claim 1, wherein the tube comprises an endotracheal tube.

3. The tube of claim 1, wherein the projection is mounted on the exterior of the body.

4. The tube of claim 1, wherein the projection comprises an oval element.

5. The tube of claim 1, wherein the projection extends outwardly from the body a distance of about 0.030 inches to about 0.080 inches.

6. The tube of claim 1, wherein the projection is integrally formed on the body of the tube.

7. The tube of claim 1, wherein the projection comprises an inflatable cuff that extends at least partially around the body of the tube.

8. The tube of claim 1, wherein the projection extends at least partially around the circumference of the body.

9. The tube of claim 1, wherein the projection comprises tubing applied to the exterior of the body.

10. The tube of claim 1, wherein the projection comprises an O-ring disposed about the body.

11. The tube of claim 1, wherein the projection extends outwardly from the body a distance of about 0.040 inches to about 0.060 inches

12. The tube of claim 1, wherein the suction opening is at least about 0.125 inches above a proximal end of the sealing cuff.

13. The tube of claim 1, wherein the projection comprises an inflatable element that is inflatable about only a portion of an annular cross-section of the body substantially orthogonal to a longitudinal axis of the gas-conveying tube.

14. A method of manufacturing a medical tube comprising:
providing a body comprising an elongated gas-conveying tube having a distal end configured to be placed in a body cavity of a patient and a proximal end configured to extend outwardly from the patient, the distal end having a distal opening and the proximal end having a proximal opening, the body having a suction lumen extending along the body and terminating in a suction opening, and wherein the suction lumen and suction opening are configured to permit suction to remove substances from the body cavity;
attaching a sealing cuff on the body proximal to the distal opening, the sealing cuff being adjacent and distal to the suction opening, wherein the sealing cuff is configured to seal against the wall of the body cavity; and
forming a projection on the exterior of the body adjacent and proximal to the suction opening, wherein the projection is configured to prevent occlusion of the suction opening and wherein the projection is configured not to seal against the wall of the body cavity.

15. The method of claim 14, wherein the body comprises an endotracheal tube.

16. The method of claim 14, wherein forming the projection on the exterior of the body comprises the use of an adhesive to secure the projection to the body.

17. The method of claim 14, wherein forming the projection on the exterior of the body comprises heating the projection to couple the projection to the body.

18. The method of claim 14, wherein forming the projection on the exterior of the body comprises securing a secondary cuff that extends at least partially around the body.

19. The method of claim 14, wherein the sealing cuff comprises an inflatable sealing cuff.

20. The method of claim 14, wherein the projection extends outwardly from the body a distance of about 0.030 inches to about 0.080 inches.

21. The method of claim 14, wherein the projection extends outwardly from the body a distance of about 0.040 inches to about 0.060 inches.

22. A method of operation for a medical tube comprising:
intubating a body cavity of a patient with a medical tube comprising:
a body comprising an elongated gas-conveying tube having a distal end configured to be placed in the body cavity and a proximal end configured to extend outwardly from the patient, the distal end having a distal opening and the proximal end having a proximal opening;
a sealing cuff mounted on the body proximal to the distal opening, wherein the sealing cuff is configured to seal against the wall of the body cavity;
a suction lumen extending along the body and terminating in a suction opening adjacent and proximal to the sealing cuff, and wherein the suction lumen and suction opening are configured to permit suction to remove substances from the body cavity; and
a projection configured to prevent occlusion of the suction opening, wherein the projection is located on the exterior of the body adjacent and proximal to the suction opening, and wherein the projection is configured not to seal against the wall of the body cavity; and
suctioning fluid via the suction lumen.

23. The method of claim 22, wherein intubating the body cavity with the medical tube comprises inflating the sealing cuff to provide a seal against the wall of the body cavity.

24. The method of claim 22, wherein the body comprises an endotracheal tube.

25. The method of claim 22, wherein the projection comprises an oblong element, wherein a longitudinal axis of the projection is substantially parallel with a longitudinal axis of the body.

26. The method of claim 22, wherein the projection comprises a ring disposed at least partially about the body.

27. The method of claim 22, wherein the projection comprises an inflatable cuff that extends at least partially around the body.

28. The method of claim 22, wherein the projection comprises a structure that is non-inflatable or adapted to substantially retain its shape during intubation.
